(19) United States
(12) Patent Application Publication
(10) Pub. No.: US 2010/0260870 A1
(43) Pub. Date: Oct. 14, 2010

(54) METHOD AND PREPARATIONS FOR TREATING INFECTION-ALLERGIC DISEASES

(75) Inventors: Elena Alexandrovna Pavlyuk, Kiev (UA); Hanna Vsevolodovna Stavysyuk, Kiev (UA)

Correspondence Address: OSHA LIANG L.L.P., TWO HOUSTON CENTER, 909 FANNIN, SUITE 3500, HOUSTON, TX, 77010, US

(73) Assignee: HANNA VSEVOLODOVNA STAVYSYUK, Kiev (UA)

(21) Appl. No.: 12/808,029

(22) PCT Filed: Dec. 21, 2007
(86) PCT No.: PCT/UA07/00079

§ 371 Date: Jun. 14, 2010

(51) Int. Cl. A61K 33/18; A61P 31/00

(52) U.S. Cl. 424/667

(57) ABSTRACT

The invention relates to medicine. The inventive method for treating infection-allergic diseases and viral infections involves the administering to a patient a preparation which is used in the form of a chemotherapeutic agent and contains the following components: 9 drops of 5% alcoholic iodine solution, 3.3 g of table salt and 0.5 1 of water. Physiotherapeutic procedures are carried out in the form of baths of an agent which contains the following components: cinnamon—3 g, the 5% alcoholic iodine solution—20 ml, table salt—250 g, water 140 1 and potato starch—25 g. In addition the patient receives as a chemotherapeutic agent for the liver probing, a preparation containing the following components: table salt 0.5 teaspoon, 9% table alcohol vinegar—1 teaspoon and boiled water 200-250 ml.

The invention relates to medicine, in particular to epidemiology, dermatology, pulmonology, gynecology, microbiology and virology, biochemistry, orthopedics, ophthalmology, cardiology, allergology, therapy, anatomy, gastroenterology, endocrinology, etc.

A great number of physicians both in past centuries and in our days has tried to gain a victory over allergic infectious diseases giving up much energy and efforts, sometimes even all the life, to this purpose. At present, doctors make use of the preparations worked out by pharmacological institutions all over the world; unfortunately, majority of them either do not help patients or even exacerbate their disease.

Let us go into detail on seven categories of medicinal agents:

Antibiotics

Anti-inflammatory agents

Antihypertensive agents

Anti-atherosclerotic agents

Antidepressants and tranquilizers

Antineoplastic agents

Vaccines, i.e. preparations aimed at introducing antigens into the organism.

What side effects of antibiotics are known?

1. They exert toxic effects on kidneys, liver, bone marrow, nervous system.

2. They induce allergy.

3. They result in intestinal upset.

4. They suppress the immune system and natural resistance of the body to infections.

5. They create predisposition to the development of malignant tumors.

Mineral corticoids. Glucocorticoids.

The therapy with corticosteroids results in various side effects:

impaired adrenal function;

elevated risk for infections;

disordered regeneration process by means of cell division;

probable appearance of contact eczema, etc.

We can read in Dr. Pavlyuk: “The adrenals simply perish following such a therapy”.

Owing to Dr. Ye.A. Pavlyuk method, all these incurable diseases can be liquidated in 2 to 3 months of the therapy under control by the hematologist and other professionals.

The implementation of Dr. Ye.A. Pavlyuk method has shown that bone marrow activity in the human organism recovers completely; no recurrences were observed during 20 years. At that, stem cell transplantation becomes unnecessary. Filtration of bone marrow goes on in the patient organism during 2-3 months under supervision by specialists and laboratory analyses.

Towards the end of therapy, such parameters, as hemoglobin, leukocytes, lymphocytes, platelets, ESR, stabs, segmented neutrophils, monocytes, and color index, confirm objectively that bone marrow is sound and ready to perform its functions in the patient organism. This is true not only for bone marrow, but also for all parts of brain.

Coordination of muscle and bone motions recovers completely as well as memory and speech. Hereupon, recovery accelerates of all organs and systems, even in patients with persistent sepsis. The question stands no longer about searching donors by blood group for patients with allergic infectious diseases, since donors are so necessary in severe traumas, accidents, and significant blood losses.

Owing to Dr. Ye.A. Pavlyuk preparations, there is no necessity in blood transfusion to clear it of infection, which exists for centuries. However, it should be noted that blood never happens without present microorganisms or viruses; a human being stays in their medium from the birth to the last breath. Moreover, numerous laboratories are working out new strains of microbes and viruses as bacteriological weapons. It should be noted that staphylococcus is the most terrible of all infections on the Earth for several millennia by now, which took away more lives than all wars taken together.

This is confirms by numerous domestic and foreign sources. One can read in Manual for General Practitioner that up to 80% of pathogenic microbes isolated from patients and staffs are, as a rule, characterized by multiresistance, not infrequently to 6-8 antibiotics. Therefore, the usage of antibiotics with prophylactic purpose does not prevent from allergic infectious diseases.

Louis Brower had written many about pharmacology and proved in detail that even restricted use of antibiotics can result, while Dr. Pavlyuk's opinion is that have already resulted, in global pandemia of infectious involvement. A new generation of microbes became ten thousand times stronger than preceding one; pharmacology has helped them to adapt to vast number of poisonous and toxic substances. In United States, 2,500 antibiotics happened to be helpless against pathogenic microbes and tumours of germinal tissues, teratoma.

By Dr. Pavlyuk's opinion, all tumour, even benign ones, should be removed, as far as, sooner or later, they transform in malignant tumours.

In medicine of all countries, there is used a great number of drugs capable of inducing pathological processes in the lungs:

Cytostatic Agents:

Bisulfane (Mizulbane, Myelosan), Cyclophosphamide (Endoxane), Karmustine (BiKNU), Lomustine.

Melfalane (ALKERAN), Chlorambucil (Chlorbutine), Mitomycin (Ameticine), Methotrexate.

Cytorabine, Vinplastine (Velbe), Procarbosine (Natulane), Bleomycine, Azathioprine (Imurane).

Antimicrobial and Antiparasitic Agents:

Nitrofurantoin (Furodonine), Furazolidone (Furoxone), Pyrimetamine+Sulfadoxine (Fansidar).

Sulfamides: Salasulfapyridine, Co-trimoxazol (Bactrime, Oriprime). Penicillins: Ampicillin (Totacillin, Penibrin). Cephalosporins: Ceftazidym, Cephalexin, Lendacin (Longaceph). Tetracyclines: Tetracycline, Metacycline (Metacycline hydrochloride). Macrolides: Erythromycin (Erythrocin).

Cardiologic Drugs:

Amyodarone (Cordarone), Nifedipine (Adalate), Persatine (Penseline), Oxprenolol (Trazicor), Propranolol (Anapryline), Pindolol (Visken), Acebutolol, Dihydralasine (Apressine), Hydrochlorothiazide (Ezidrex).

Rheumatologic Drugs

Gold drugs (Auropane), Penicillamine (Kuprenil), Naproxen (Naprosine), Sulindac, Ibuprofen (Brufen), Dantrolen (Dantrium).

As revealed by Dr. Ye.A. Pavlyuk, drugs containing barbiturates occurred to be most dangerous:

Valocarmide, Valocordin, Corvalol, Corvaldine, etc.

Neurological Drugs:

Mezheptil (Thioproperasine), Dihydroergotamine (Ditamine), Bromocriptine (Parlodel), Chlorpromazine, Imipramine (Tofranil), Carbamazepine (Tegretol), Phenytoin, Maliazine (Barbexaclone), Dosulepine.

Drugs Exerting Influence on Metabolism:

Chlopropamide (Diabamide), Clofibrate (Misklerone), Propylthiouracyl.

Anti-Allergic Drugs:

Beclometazone (Becotide), Cromoglycine (Lomuzol).

Used in the current medicine are magnetotherapy, laser therapy, AZT-therapy, electromagnetotherapy, acupuncture, radical surgical interventions, etc. However, these therapeutic modalities did not help to solve the problem of spreading allergic infectious diseases.

Dr. Ye.A. Pavlyuk's method has been developed specifically to put an end to epidemics and determine the spreading of allergic infectious diseases over the world. It would be desirable that UN and UNICEF exerted support to implementation of this method.

According to Dr. Ye.A. Pavlyuk's method, the prescriptions are as follows:

1. Lavage of the universal nidus of infection with a Dr. Ye.A. Pavlyuk's preparation, <<Zhiznedan>>.

2. Blood filtration with the use of <<Zhiznedan>> and “Silodanochel” preparation.

3. Intake of <<Zhiznedan>> preparation, but with granatum or lemon juice added.

4. Recovery of affected organs or skin with the use of physiotherapeutic procedures and “Epizdorovir” preparation. The list of diseases, which can be cured and prevented is sufficiently long, over 300 items.

According to Dr. Ye.A. Pavlyuk's method, there have been developed methods for the treatment of all varieties of cancer as well as the following diseases:

American encephalitis

West Nile encephalitis

Australian encephalitis

Californian encephalitis

Murray Valley encephalitis

Venezuela horse encephalitis

Tropical Mosquito Fevers:

Bwamba fever

Semlica fever

Bunyavir fever

Bunyavir fever

Uganda fever

Hemorrhagic Fevers:

Crimean hemorrhagic fever

Manchurian hemorrhagic fever

Hemorrhagic nephrosis/nephritis

Korean hemorrhagic fever

Bukovyna hemorrhagic fever

Swiss hemorrhagic fever

Puerto-Rican seven-day fever

Indian seven-day fever

Indian three-day fever

Bulgarian hemorrhagic fever

Autumn fever A

Autumn fever B

US Coxsackie disease

Hebr impetigo herpetiformis

Blennorrheal keratosis

Chronic lupus erythematosus

Acute lupus erythematosus

Rubeola scarlatinosa

Infectious erythema

Exanthem subitum

Gastroenterrhagic fever of Tunisia

Onyalai

Finally, Ebola virus disease, which threatens by global epidemic.

Lass hemorrhagic fever

Marburg hemorrhagic fever

Hemorrhagic fever with the renal syndrome (HFRS)

Yellow fever

With the use of Dr. Ye.A. Pavlyuk's method, the following infectious diseases can be treated:

Adenoviral infection

Amebiasis

Ankylostomiasis and necatoriasis

Rabies

Creutzfeldt-Jakob disease (mad cow disease)

Cat-scratch disease (benign giant follicular lymphoblastoma)

Typhoid fever

Botulism

Brucellosis

Spirochetosis inverse (mite-borne)

Relapsing fever, epidemic

Viral hepatitis type A

Hepatitis type B

Hepatitis type C

Hepatitis type E

Herpetic infection

Influenza

Dysentery

Diphtheria

Yersiniasis

Haemophylus influenzae infection

Campylobacteriosis

Clebsiellosis

Pertussis

Coli-infection (escherichiosis, colienteritis)

Measles

Q fever

Leprosy

Lyrnphocytic choriomeningitis (phlebotomus fever, mosquito)

Tetanus

Cholera

Other Mosquito Viral Infections:

Rift Valley fever

Mite-borne fever

Mengo meningitis

Infectious adenitis

Infections of External Coverings (Percutaneous Infections):

Pemphigus (tropical)

Dermatitis herpetiformis (Duhring's disease)

Infectious warts

Oral mucosal papillomas

Plantar warts

Virus of molluscum contagiosum

Smallpox and Smallpox-Like Diseases:

Sheep-pox in humans

Eczema vaccinatum

Parapsoriasis

Keratosis follicularis, contagious

Seborrheic eczema

Dermatitis exfoliativa neonatomm, epidemic

Tropical ulcer

Ulcer of deserts

Acrodermatitis vesiculosa tropica

Mirabelli parakeratosis

Brazilian pemphigus foliaceus

Asbestos-like epidermodermitis

Papuliferous pseudolues

Follicular dermatosis, epidemic

Neuro Infections:

Jilas Valley meningoencephalitis

Encephalomeningitis

Primary infectious polyradiculoneuritis—subacute progressive poliomyelitis

Acute seasonal serous meningitis

Encephalomyelitis following antirabic prophylactic immunizations

Primary encephalitis with mental disorders

Infectious encephalitis

Special form of children's viral infection

Chronic poliomyelitis

Optico

Hemorrhagic leukoencephalitis

Lukyanchikov syndrome

Mild leukocytic meningitis

Benign recurrent meningitis

Hemorrhagic leukoencephalitis

Encephalitis with facial nerve affected

Lumbosacral radiculitis

Myelopolyradiculitis

Polyneuritis, epidemic

Mononeurites

Landry ascending paralysis

Infectious pachymeningitis

Pseudopoliomyelitis in Island

Chorea

Thoracocervical radiculitis

Subacute necrotic myelitis

In the Netherlands, euthanasia is permitted by law. It seems that other countries will suffer the same fate, if prompt actions will not be taken to implement Dr. Ye A. Pavlyuk method.

According to this method, a roentgenogram of accessory sinuses of the patient's nose is made as well as a bacteriological examination for pathogenic flora. For that purpose, smears are taken from the patient's pharynx and nose separately into two test tubes, and inoculation of agar medium is made to test them for growth and resistance to antibiotics.

Then, according to the method indicated, the patients are prescribed concurrently:

1. To treat allergic infectious and viral diseases, the patient is prescribed the <<Zhiznedan>> preparation as a chemotherapeutic drug. The preparation indicated consists of 5% alcohol solution of iodine—9 drops, table salt—3.3 g, and water at the temperature of +18° C.—0.5 L.

2. Irrigation of the patient's nasopharynx with the <<Zhiznedan>> preparation is prescribed as a physiotherapeutic procedure.

3. Peroral administration of the <<Zhiznedan>> preparation with 1 drop of pomegranate or lemon juice added per liter by 1-2 swallows daily during all the 2-3 month course of therapy (see Claim 1).

4. To ensure blood filtration, the <<Zhiznedan>> preparation is administered as 5-ml injections i.m. prior to meal once a day. A course of the therapy consists of 15 injections with 2-day interval.

5. Irrigation of the patient's urethra using a 125- to 250-ml syringe with the same pharmacological composition <<Zhiznedan>>:

| | | |
|---|---|---|
| 5% alcohol solution of iodine | 9 | drops |
| table salt | 3.3 | g/L |
| water at the room temperature | 0.5 | L |

6. In case of skin diseases, cold compresses with the <<Zhiznedan>> preparation are prescribed onto the skin areas affected with rash, abscesses, ulcers, tumors, swellings or stings (according to Claim 1).

7. Bathes are prescribed as a physiotherapeutic procedure, and “Epizdorovir” preparation comprising cinnamon—3 g, 5% iodine alcohol solution—20 ml, table salt 250 g, boiled water 140 1, potato starch—25 g water is used as a chemotherapeutic agent.

8. Hepatic “probing” is prescribed as a physiotherapeutic procedure. For that purpose, “Silodanochel” preparation comprising table salt—half of a tea spoon, 9% table vinegar—one tea spoon, boiled water—200 to 250 ml.

The preparation is taken prior to meal; neither a hot-water bag, nor a probe has to be used. A course of the therapy lasting 2-3 months at any gastric acidity is the following: intake of the preparation—two weeks, interval—one week, intake of the preparation—one week. One course of the therapy ensures complete recovery of the liver, even when its sizes are enlarged +2, +3, +4, +5, +6, +7, in cirrhosis and metastatic disease.

9. The patient's nutrition should be of full value. Sour, spicy and salty foodstuffs are mandatory items as well as brines and sour-sweet juices.

### EXAMPLE 1

Female patient, Lydia, 65 years, diagnosis: staphylococcus, maxillary sinusitis, rheumatism, IHD in active phase, furunculosis; development of viral infection as a result of mosquito stings. Hemoglobin 95, lymphocytosis, protein in urine. Diabetes mellitus.

Following the therapy conducted out, the patient's state has been normalized: sugar—within the normal limits; liver—enlargements are neither on the left, nor on the right lobes. ECG—conductivity of both the ventricles is normal. Protein is not revealed in urine. Skin coverings are clear. The bacteriological examination of nasopharynx did not revealed staphylococci.

### EXAMPLE 2

Female patient, Tanya, 25 years, diagnosis: vegeto-vascular dystonia. Inflammation of uterine appendages. ESR—60. Gall bladder is enlarged, contains concrements. Pancreatitis. The patient has had Botkin's disease. Two nodes are revealed in the thyroid gland.

Following the therapy conducted out, the patient is healthy. ESR—normal, leukocytic formula—normal. Inflammation of uterine appendages is not determined. AP-127/80 mm Hg. USE shows normal after a great amount of sand and pus has been removed.

### EXAMPLE 3

Female patient, Aliya, 53 years, diagnosis: trigeminal neuralgia, often acute respiratory infections, arthritis; the patient is incapable to walk along the room. Fever is noted during 5 days. Erythemas have appeared on the body, throat is hyperemic. Deglutitive problem. Swollen extremities. Ethmoiditis. Prior to the therapy, staphylococcus was cultured from the patient's nasopharynx. Virus of yellow fever was revealed in blood; blood was revealed in urine.

Following the therapy conducted out, the patient's state has been normalized, mucosa became cleared, nutrition—normal. Swallowing is recovered. The patient is able to walk; inflammatory swelling of the joints is absent. Repeated examinations did not reveal pathogenic staphylococcus. Trigeminal neuralgia was cured completely.

### EXAMPLE 4

Female patient, Lena, 34 years, diagnosis: mammary lymphosarcoma, bronchial asthma, acute cardiac failure, parapsoriasis. Hb 70.

Following the therapy conducted out, the patient's state has been normalized, lymphosarcoma is no longer revealed, and conductivity of ventricles and atria became normalized. State of the bronchial tree is normal, and asthmatic attacks are discontinued.

### EXAMPLE 5

Female patient, Tatiana, 56 years, diagnosis: periauricular lymphadenitis on the right transformed in lymphosarcoma, fever, body temperature of 39° C. Vaginal peduncular polyp transformed in tumor. X-ray of the accessory sinuses of nose showed pansinusitis. Staphylococcus aureus was cultured from the patient's nasopharynx.

Following the therapy conducted out, the patient's state has been normalized. The peduncular polyp has fallen off. The accessory sinuses of nose have no longer opacities. The immunogram showed normal.

### LITERATURE

1. Peter D. Adamo, Katrina Whitney. A treatment-and-prophylactic program for 4 groups of blood.

2. Ed. Bunin K. V. & Zakstel'skaya L. Ya. Infectious diseases.

3. AcRAS & RAMS, A. I. Vorobyova. Manual for general practitioner.

4. V. M. Bergol'tz, N. S. Kislyuk, V. S. Yeremeyev. Immunology and immunotherapy of leucosis.

5. V. V. Men'shikova, N. I. Volkova. Biochemistry.

6. Ye. M. Tareyev. Internal diseases.

7. A. S. Troshin, Yu. B. Vakhtin, A. D. Brown, L. N. Zhinkin, K. M. Sukhanova. Cytology.

8. Ye. V. Schmidt, N. V. Vereshchagina. Manual on neurology.

9. L A. Alov, L A. Broude, M. Ye. Aspiz. Fundamentals of cell functional morphology.

10. D. Mezia. Mitosis and physiology of cell division.

11. D. Poll. Culture of cells and tissue.

12. E. Pirs. Histochemistry.

13. E. Roberts, V. Novinsky, F. Saes. Biology of the cell.

14. G. M. Frank. Living cell.

15. G. M. Frank (Ed.). Molecules and cells.

16. A. D. Ado & L. M. Ishimova. Pathological physiology.

17. Alliance. Book for those not indifferent. Human beings and HIV.

1. A method for treatment of infectious-allergic diseases or viral infections,
which method comprises administering to the patient or applying onto a body of the patient a chemotherapeutic preparation in a course of 2-3 months, wherein a composition of the preparation is the following:

| | |
|---|---|
| 5% alcohol solution of iodine | 12 to 18 drops per liter |
| table salt | 6.6 g/L |
| water at 12 to 15° C. | qs to 1 L |

2. The method according to claim 1, characterized in that the patient is prescribed the irrigation of nasopharynx with the preparation three times a day prior to meal.

3. The method according to claim 1, characterized in that 3 drops of granatum juice are added to the preparation and the preparation is administered orally by 1-2 swallows daily during all the 2- or 3-month course of therapy.

4. The method according to claim 1, characterized in that the preparation to the patient is administered intramuscularly as 5-ml injections prior to meal once a day with total course of the therapy consisting of 15 injections with 2-day interval following each 5 injections, except the case of chroniosepsis, when a course of the therapy consists of 75 i.m. injections consisting of five 5-ml injections per day.

5. The method according to claim 1, characterized in that the patient is administered to the patient intravenously as 5-ml injections prior to meal once a day, in the morning, with a total course of the therapy consisting of 15 injections.

6. The method according to claim 1, characterized in that the patient is prescribed the irrigation of urethra with the preparation using a 125-250-ml syringe without a needle of a “\check{Z}ene” type or an enema of the same volume.

7. The method according to claim 1, characterized in that the patient is prescribed cold compresses as physiotherapeutic procedure with the preparation, to which 3 g of pepper are added optionally in severe cases, onto the skin areas affected.

8. The method according to claim 1, characterized in that the patient is prescribed as a physiotherapeutic procedure bathes with the preparation, to which cinnamon—3 g, potato starch—25 g and acacia tincture—3 g are added to 140 liters of water, with a total course of the therapy consisting of 20 bathes with 2- or 3-day interval.

9. The method according to claim 1, characterized in that the patient is prescribed the hepatic “probing” prior to morning meal with a use of a solution comprising:

| | |
|---|---|
| table salt | half tea-spoon |
| 9% table vinegar | one tea-spoon |
| boiled water | 200 to 250 ml |

10. The method according to claim 1, characterized in that the patient is prescribed nutrition of full value, which should daily include a great amount of sour, spicy and salty foodstuffs and 100 g of preserved vegetable brine.

11. The method according to claim 2, characterized in that the patient is prescribed the hepatic “probing” prior to morning meal with a use of a solution comprising:

| | |
|---|---|
| table salt | half tea-spoon |
| 9% table vinegar | one tea-spoon |
| boiled water | 200 to 250 ml |

12. The method according to claim 3, characterized in that the patient is prescribed the hepatic “probing” prior to morning meal with a use of a solution comprising:

| | |
|---|---|
| table salt | half tea-spoon |
| 9% table vinegar | one tea-spoon |
| boiled water | 200 to 250 ml |

13. The method according to claim 4, characterized in that the patient is prescribed the hepatic “probing” prior to morning meal with a use of a solution comprising:

| | |
|---|---|
| table salt | half tea-spoon |
| 9% table vinegar | one tea-spoon |
| boiled water | 200 to 250 ml |

14. The method according to claim 5, characterized in that the patient is prescribed the hepatic “probing” prior to morning meal with a use of a solution comprising:

| | |
|---|---|
| table salt | half tea-spoon |
| 9% table vinegar | one tea-spoon |
| boiled water | 200 to 250 ml |

15. The method according to claim 6, characterized in that the patient is prescribed the hepatic “probing” prior to morning meal with a use of a solution comprising:

| | |
|---|---|
| table salt | half tea-spoon |
| 9% table vinegar | one tea-spoon |
| boiled water | 200 to 250 ml |

16. The method according to claim 7, characterized in that the patient is prescribed the hepatic “probing” prior to morning meal with a use of a solution comprising:

| | |
|---|---|
| table salt | half tea-spoon |
| 9% table vinegar | one tea-spoon |
| boiled water | 200 to 250 ml |

17. The method according to claim 8, characterized in that the patient is prescribed the hepatic “probing” prior to morning meal with a use of a solution comprising:

| | |
|---|---|
| table salt | half tea-spoon |
| 9% table vinegar | one tea-spoon |
| boiled water | 200 to 250 ml |

18. The method according to claim 2, characterized in that the patient is prescribed nutrition of full value, which should daily include a great amount of sour, spicy and salty foodstuffs and 100 g of preserved vegetable brine.

19. The method according to claim 3, characterized in that the patient is prescribed nutrition of full value, which should daily include a great amount of sour, spicy and salty foodstuffs and 100 g of preserved vegetable brine.

20. The method according to claim 4, characterized in that the patient is prescribed nutrition of full value, which should daily include a great amount of sour, spicy and salty foodstuffs and 100 g of preserved vegetable brine.
